(19) United States
(12) Patent Application Publication
(10) Pub. No.: US 2016/0313635 A1
(43) Pub. Date: Oct. 27, 2016

(54) MEDICAL IMAGING APPARATUS AND PROJECTION DEVICE

(71) Applicant: KABUSHIKI KAISHA TOSHIBA, Tokyo (JP)

(72) Inventors: Takashi SASAKI, Yokohama (JP); Aira Hotta, Kawasaki (JP); Kazuo Horiuchi, Yokohama (JP); Sayaka Takai, Yokohama (JP); Tomoya Tsuruyama, Kawasaki (JP); Shinichiro Koto, Tokyo (JP); Hisao Tanaka, Yokohama (JP)

(21) Appl. No.: 15/131,853

(22) Filed: Apr. 18, 2016

(30) Foreign Application Priority Data
Apr. 21, 2015 (JP) 2015-087126

(51) Int. Cl. G03B 21/53; A61B 6/03; G03B 21/28; A61B 5/055; G03B 21/12; G03B 21/14

(52) U.S. Cl. CPC G03B 21/53; G03B 21/12; G03B 21/142; G03B 21/28; A61B 5/055; A61B 6/032

(57) ABSTRACT

A medical imaging apparatus comprises a gantry, a table, a first emitting unit, and a reflection unit. The table is movable to go in and out of the gantry. The first emitting unit is apart from the gantry and configured to emit image light. The reflection unit is configured to reflect the image light toward the table.

### CROSS-REFERENCE TO RELATED APPLICATIONS

This application is based upon and claims the benefit of priority from Japanese Patent Application No. 2015-087126, filed Apr. 21, 2015, the entire contents of which are incorporated herein by reference.

### FIELD

Embodiments described herein relate generally to medical imaging apparatus and to projection devices.

### BACKGROUND

In recent years, projection devices have been considered for use in various fields. For example, projection devices have been considered for use in medical imaging devices, to display still images and video images.

### BRIEF DESCRIPTION OF THE DRAWINGS

FIG. 1 is a schematic view illustrating a projection device according to one embodiment.

FIG. 2A is a schematic view illustrating a state of a nuclear magnetic resonance imaging device in which a table is outside of a gantry.

FIG. 2B is a schematic view illustrating a state of a nuclear magnetic resonance imaging device in which a table is inside of a gantry.

FIG. 3A is another schematic view illustrating a state of a nuclear magnetic resonance imaging device in which a table is outside of a gantry.

FIG. 3B is another schematic view illustrating a state of a nuclear magnetic resonance imaging device in which a table is inside of a gantry.

FIG. 4 is a schematic view illustrating a projection device according to another embodiment.

FIG. 5 is a flow chart illustrating a method performed by a projection device.

### DETAILED DESCRIPTION

Each of the embodiments will now be described in detail with reference to the accompanying drawings.

Note that the figures are conceptual pattern diagrams, and the relationships between thicknesses and widths and ratios of size of each part are not necessarily represented to scale. Moreover, the size and ratio of components that appear in multiple figures are not necessarily the same in each figure.

Note also that in the following embodiments, parts denoted by the same reference numerals are assumed to operate similarly, and a repetitive description thereof will be omitted, as appropriate.

According to one embodiment, a medical imaging apparatus comprises a gantry, a table (or gurney), a first emitting unit, and a reflection unit. The table is movable to go in and out of the gantry. The first emitting unit is apart from the gantry and configured to emit image light. The reflection unit is configured to reflect the image light toward the table.

According to another embodiment, a projection device comprises a first emitting unit. The first emitting unit is configured to emit image light toward a movable table. The first emitting unit is configured to change an image location of the image light based on a position of the table.

According to one embodiment, a medical imaging apparatus comprises a projection device, a gantry, and a table. The projection device comprises a first emitting unit. The first emitting unit is configured to emit image light toward a movable table. The first emitting unit is configured to change an image location of the image light based on a position of the table. The table is configured to go in and out of the gantry.

According to one embodiment, a projection device is movable to follow a table on which an observer is mounted and is configured to emit image light.

### First Embodiment

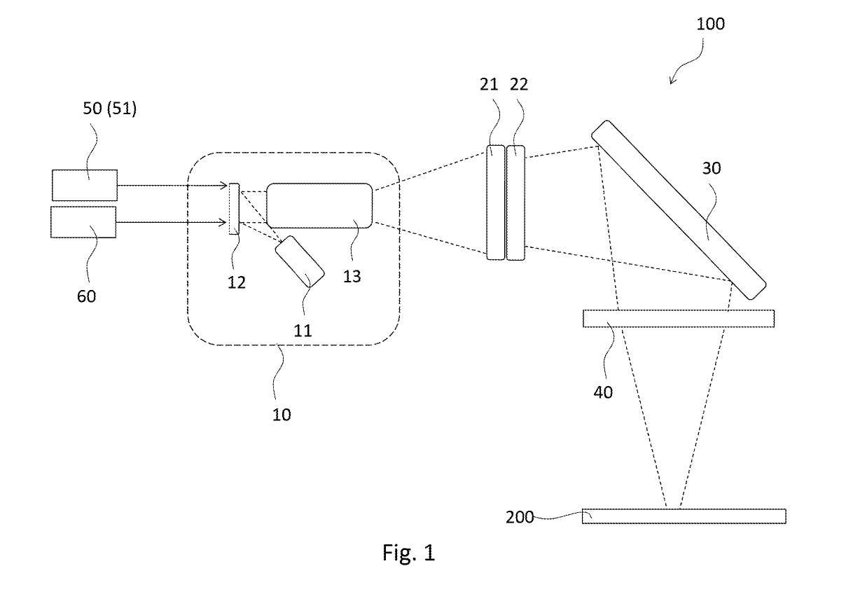

FIG. 1 is a schematic view illustrating a projection device 100. The projection device 100 is movable to follow a movement of a table on which an observer is mounted. The projection device 100 emits image light.

The projection device 100 comprises a first emitting unit 10 which emits image light toward the movable table. The first emitting unit 10 is configured to change an image location of the image light based on a position of the table 200. That is, the first emitting unit 10 is configured to change an image location of the image light based on a position of an observer mounted on the table.

The projection device 100 may project a video image on a projection plane. That is, an image explained in this embodiment may be a still image or a video image.

The first emitting unit 10 may, for example, comprises a display element 12 and a first optical element 13. The display element 12 displays an image based on an image signal. The first optical element 13 images light (light rays) from the display element 12 on a determined location. The first optical element 13 may comprise an optical element such as lens and/or prism. The first optical element 13 may comprise a plurality of optical elements. The first emitting unit 10 may further comprises a light source 11. The light source emits light toward the display unit 12.

For example, the farther the distance from the first emitting unit 10 to the table 200 is, the farther the first optical element 13 may be able to form an image location. The first emitting unit 10 may be movable and change the image location by moving based on the position of the table 200. The first emitting unit 10 may comprise a lens (or a plurality of lenses) configured to change an image location, e.g., by moving a position (or positions) of the lens (or the lenses) so as to change a focal length of the optical element. The first optical element 13 may comprises the lens or the plurality of lenses.

For example, the first emitting unit 10 (the first optical element 13) may comprise a plurality of lenses whose focal lengths are different from each other. One or some of the lenses may be provided on the light path of the image light and the other(s) may not be provided on the light path. The first emitting unit 10 may change an image location by changing an arrangement of the lens (or lenses) provided on the light path. By changing an arrangement of the lens (or lenses) provided on the light path of the image light, a focal point is changed.

By this embodiment, an image which is in focus is provided to an observer since an image location of the image light is able to be changed based on a position of the table 200.

The projection device 100 may further comprise a first position acquiring unit 50 to acquire first information regarding a position of the table 200. The first information may, for example, include information of a position of the table 200 which is expressed with reference to the position of the projection device 100 or the first emitting unit 10. Relative positional relation between a projection unit and the table 200 may be determined. The image light from the first emitting unit 10 is projected on the projection unit. The projection unit will be described later. That is, the projection device 100 is configured to acquire the position of a projection unit by acquiring the position of the table 200. Therefore, the first emitting unit 10 is able to change the image location to be located on the projection unit.

The first information may be input from the table 200 to the first position acquiring unit 50. The first information may be input to the first position acquiring unit 50 by a sensor included in the projection device 100 to detect a position of the table 200. The first information may be manually input to the first position acquiring unit 50 with an input unit such as a keyboard and a mouse.

The projection device 100 may comprises a second position acquiring unit 51 instead of the first position acquiring unit 50. The second position acquiring unit 51 acquires second information regarding a position of a reflection unit 30 arranged on a light path of the image light between the table 200 and the first emitting unit 10. The relative positional relation between the projection unit and the reflection unit 30 is determined. That is, the projection device 100 is configured to acquire the position of the projection unit by acquiring the position of the reflection unit 30. That is, the first emitting unit 10 is able to change the image location to be located on the projection unit.

The second information may, for example, include information of a position of the reflection unit 30 expressed with reference to the position of the projection device 100 or the first emitting unit 10. The second information may be input to the second position acquiring unit 51 by a sensor included in the projection device 100 to detect a position of the reflection unit 30. The second information may be manually input to the second position acquiring unit 51 with an input unit such as a keyboard and a mouse.

The projection device 100 may further comprise an image signal acquiring unit 60 to acquire an image signal. In this case, the first emitting unit 10 emits image light based on the image signal.

The projection device 100 may further comprise the reflection unit (light reflection unit) 30. The reflection unit 30 may be, for example, on a light path of the image light between the first emitting unit 10 and the table 200. The reflection unit is light reflective and reflects at least a part of the image light emitted from the first emitting unit 10 toward the table 200. The reflection unit may, for example, have light reflectivity of more than 70 percent. The reflection unit 30 may, for example, comprise a light reflective plane. The light reflective plane may, for example, comprise a material including a dielectric multilayer film or metal. The reflection unit 30 may further have refractivity. The reflection unit 30 may, for example, be configured to adjust an angle of the light reflective plane.

The projection device 100 may further comprise a projection unit 21. The projection unit 21 may be a light transmittable screen. The projection device 100 may further comprises a second optical element 22. The projection unit 21 and the second optical element 22 may be arranged on a light path of the image light between the first emitting unit 10 and the table 200. The projection unit 21 may, for example, be arranged on a light path of image light between the first emitting unit 10 and the reflection unit 30. The second optical element 22 may, for example, be arranged between the projection unit 21 and the reflection unit 30.

The projection unit 21 may be provided on the image location formed by the first emitting unit 10. The distance between the projection unit 21 and the image location may be not more than 10 percent of the distance between the first emitting unit 10 and the image location. A real image formed by light (light rays) emitted from the first emitting unit 10 is projected on the projection unit 21.

For example, the second optical element 22 may transmit at least a part of the light from the first emitting unit 10. For example, the second optical element 22 may change a travel direction of light. For example, the second optical element 22 may enlarge or reduce the image on the projection unit 21.

Light reflected by the reflection unit 30 may travel toward the table 200 which is an observation position. The reflection unit may reflect a part of light and refract another part of light. An observer may observe an image projected on the projection plane. For example, the first emitting unit 10, the projection unit 21, and the reflection unit 30 may be arranged along a first direction. The reflection unit 30 and the table may be arranged along a second direction that crosses the first direction. An angle formed by the first angle and the second angle is larger than 0 degree and smaller than 180 degrees. For example, the angle may be 90 degrees.

A relative positional relation among the projection unit 21, the second optical unit 22, and the reflection unit 30 is determined. The projection unit 21, the second optical unit 22, and the reflection unit 30 may be fixed relative each other.

For example, to move an image location, the first emitting unit 10 is moved to follow a movement of the projection unit 21, the second optical unit 22, and the reflection unit 30. That is, a direction and distance of movement of the projection unit 21, the second optical unit 22, and the reflection unit 30 corresponds to the direction and distance of movement of the first emitting unit 10.

An observer mounted on the table 200 is able to observe a real image on the projection unit 21. In the case where the second optical element 22 enlarges light rays, an observer is able to observe an image larger than the image on the projection unit 21. In the case where the reflection unit 30 has a refractive power, an observer is able to observe an image further enlarged.

The projection device 100 may further comprise an ocular optical unit 40. The ocular optical unit 40 is, for example, arranged between the reflection unit 30 and the table 200. The ocular optical unit 40 may be, for example, arranged to form a virtual image on the position based on the visual power of an observer. The ocular optical unit 40 may have a power of concentration of light. The ocular optical unit 40 may be lens.

It is preferred that an image height (a size of a real image on the projection unit 21) does not change in the case where an image location is changed by the first emitting unit 10 following the position of the table 200. The first emitting unit 10 may be, for example, configured to change the image location so that an image height after the change is not less than 90 percent and not more than 110 percent of the image height before the change.

For example, the first optical element 13 may be configured to form an image larger than an image on the display element 12. By the projection device 100, it is configured to provide a wide-field image to an observer.

Brightness of an image on the projection unit 21 is proportional to the square of a distance between the first emitting unit 10 and an image location. Therefore, luminance of the light source 11 or the display element 12 may be adjusted based on the distance. That is, it is preferred that the longer the distance between the first emitting unit 10 and an image location is, the higher the luminance of an image light from the first emitting unit 10.

By the projection device 100 according to this embodiment, it is possible to provide an image which is in focus to an observer even while an observation position changes. That is, the focus and image height of an image formed by the projection device 100 is kept even during the time in which the observation position of the observer changes.

### Second Embodiment

In this embodiment, a medical imaging apparatus comprising the projection device 100 will be described. A nuclear magnetic resonance imaging apparatus (MRI apparatus) 500 will be described as an example of a medical imaging apparatus. However, the medical imaging apparatus may be another type of medical imager, for example, a computed tomography apparatus.

MRI apparatuses are used to acquire a section image of a patient non-invasively by magnetic resonance. To acquire a section image, a patient is put in a tubular gantry comprising a magnetic coil. In such a closed environment, a patient needs to endure a cooped-up feeling and stress. It is desirable to calm the cooped-up feeling and stress of a patient who has claustrophobia.

As a countermeasure to alleviate a cooped-up feeling and stress, a technique has been proposed in which an image is projected on the wall of examination room when a table on which the patient is mounted enters a gantry. The image is provided to the patient by a mirror arranged in front of the patient. With this technique, however, it is difficult to avoid the patient visually recognizing the fact that they are entering into the gantry.

The nuclear magnetic resonance imaging apparatus 500 comprises the first emitting unit 10, the reflecting unit 30, a gantry 301, and a table 302. The gantry 301 includes outer wall 311 and inner wall 312. The table 302 may be, for example, configured to enter the inside of the inner wall 312.

The nuclear magnetic resonance imaging apparatus 500 may comprise the projection device 100, the gantry 301, and the table 302.

In this embodiment, the nuclear magnetic resonance imaging apparatus 500 comprising the gantry 301, the table 302, and the projection device 100 including the first emitting unit 10, the reflection unit 30, the first acquiring unit 50 (or the second acquiring unit 51), the image signal acquiring unit 60, and the second optical element 22 will be described.

The projection device 100 may, for example, comprise the projection unit 21 which is configured to move to follow the movement of the table 302. The size of the projection unit 21 is smaller than an image to be projected on the projection unit 21. By leaked light from the peripheral edge of the projection unit 21, an image with high-realistic sensation may be provided. A part of the leaked light is projected on the inner wall 312 of the gantry 301.

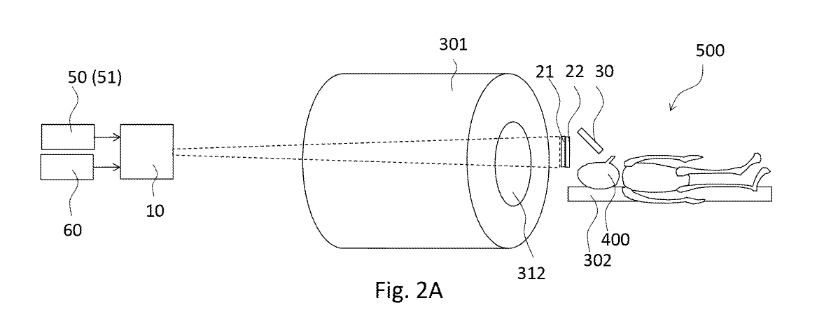

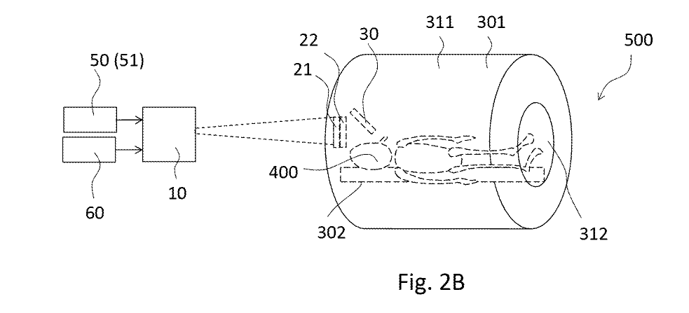

An example of a means to change an image location by the first emitting unit 10 will be described. FIG. 2B is a schematic view illustrating a state of a nuclear magnetic resonance imaging device which a table is inside of a gantry.

A relative positional relation among the projection unit 21, the second optical element 22, and the reflection unit 30 is fixed by supporting components. An observer 400 is mounted on the table 302. The table 302 is configured to go in and out of the gantry 301. The position of the emitting unit 10 is fixed outside of the gantry 301.

A position of the table 302 is decided based on a part of a patient (who is also an observer observing an image) to be imaged by the nuclear magnetic resonance imaging apparatus 500. That is, the table 302 is placed so that the part to be imaged is arranged at or near the center of the gantry 301.

The first emitting unit 10 is able to move a focal point to follow a position of the observer on the table 302. Therefore, it is able to provide a clear and unblurred image in focus to an observer even while the table moves.

By the projection device 100 and the nuclear magnetic resonance imaging apparatus 500 in this embodiment, a wide-field image is provided in focus even when an observation position changes. That is, the focus and image height of an image formed by the projection device 100 is kept even while an observation position changes.

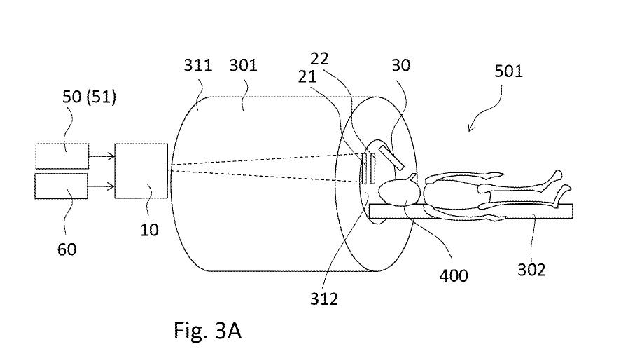

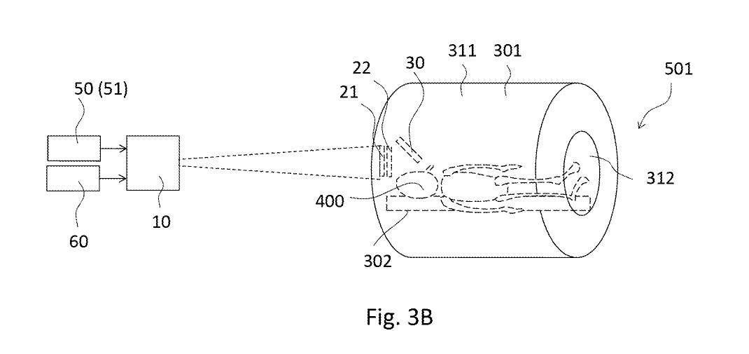

Another example of a means to change an image location by the first emitting unit 10 will be described. FIG. 3A is a schematic view illustrating a state of a nuclear magnetic resonance imaging device in which a table is outside of a gantry. FIG. 3B is a schematic view illustrating a state of a nuclear magnetic resonance imaging device in which a table is inside of a gantry.

In this embodiment, the first emitting unit 10 is arranged apart from the gantry 301. The first emitting unit 10 is configured to move to follow the movement of the table 302 on which the observer is mounted. That is, a relative positional relation among the first emitting unit 10, the projection unit 21, the second optical element 22, the reflection unit 30, and the table 302 is fixed by a supporting component. The projection unit 21, the second optical element 22, the reflection unit 30, and an observer 400 are able to go in and out of the gantry 301 following the movement of the table 302. The first emitting unit 10 is configured to move to follow the movement of the table 302. That is, it is able to provide a clear and unblurred image to an observer by the projection device even while the table 302 moves.

A strong magnetic field is generated in the gantry 301 of the nuclear magnetic resonance imaging apparatus 500. It is preferred to avoid arranging a component which easily influences a magnetic field near the gantry 301 since the body signals that the nuclear magnetic resonance imaging apparatus 500 detects are weak.

The first emitting unit 10 is arranged outside of the gantry 301 in this embodiment. The first emitting unit 10 is close to the gantry 301 when the table 302 is outside of the gantry 301, and the first emitting unit 10 is far from the gantry 301 when the table 302 is inside of the gantry 301. Therefore, the nuclear magnetic resonance imaging apparatus 500 is less influenced by the first emitting unit 10 when a magnetic field is generated in the gantry 301. The distance between the first emitting unit 10 and the gantry 301 is preferably more than two meters. It is preferred that a shield component is arranged between the first emitting unit 10 and the gantry 301 to shield a magnetic field.

In the case where the projection unit 21 is small, it is easy for an observer to feel pressured by the inner wall 312 of the gantry since the inner wall 312 is visible from the observer directly. When the table 302 goes into the gantry 301, the entry into the gantry is easily recognized by the observer.

Therefore, it is preferred that shapes of the reflection unit 30 and the projection unit 21 are the same as or similar to a cross-sectional shape of inner wall 312 of the gantry 301. The cross-section is perpendicular to the direction in which the tubular gantry 301 extends. The projection unit 21 is, for example, apart from the inner wall 312 to allow for air to blow between the projection unit 21 and the inner wall 312. The distance between a peripheral edge of the projection unit 21 and the inner wall 312 is, for example, not more than 10 centimeters, preferably not more than 5 centimeters, more preferably not more than 2 centimeters.

A viewing angle of an image to be observed by the observer is preferably 60 degrees so that it is difficult for the observer to recognize the inner wall 312. That is, a distance L between the projection unit 21 and an eye of an observer through the reflection unit 30 along a light path is preferable to be not more than a screen radius of the projection plane/tan (30 degrees). Furthermore, the distance between the projection unit 21 and an eye of an observer along a light path is preferably not less than the radius of the cross-section of the inner wall 312.

By the projection device 100 and medical imaging device according to this embodiment, it is possible to provide an image which is in focus to an observer even when an observation position changes.

### Third Embodiment

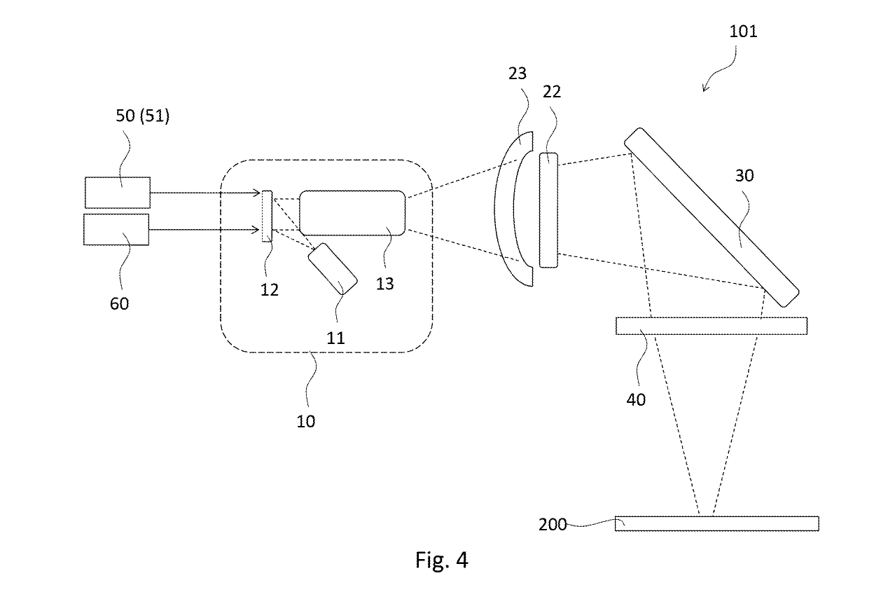

A projection device 101 comprising a projection unit including curved surface will be described in this embodiment. FIG. 4 is a schematic view illustrating a projection device according to a third embodiment.

A projection unit 23 includes a curved surface convex with respect to the first emitting unit 10. The curvature of the curved surface is, for example, not less than a half of the width of the cross-section (a radius) of the inner wall 312 and not more than the width of the cross-section (a diameter) of the inner wall 312. In the case where the cross-section of the inner wall 312 is not a circle, the width of the cross-section of the inner wall 312 may be an average of the widths of the inner wall 312.

In the case where the projection unit 32 includes the curved surface, a wide field-of-view is able to be provided to an observer.

A projection unit 21 including a plane surface and a projection unit 23 including the curved surface will be compared to each other in a situation where the distances between an eye of an observer and each the projection units 21, 23 are the same. It is found by the inventors that an image on the projection unit 23 including the curved surface gives the observer an impression of a sense of depth and width.

In the case where the curvature of the curved surface is too small, an observer easily recognizes the inner wall 312 when the table 302 enters the gantry 301, since the projection unit 23 is far from the inner wall 312. In the case where the curvature of the curved surface is too large, it is difficult to obtain the visual effect of curved surface.

The center part of an image on the projection unit 23 may appear to be too light. It is preferable to adjust the projection device 101 so that the brightness of the image becomes uniform. The means to adjust the brightness may include correcting the image signals, controlling the distribution of a transmission of the projection unit 23, and correcting the brightness of image light from the first emitting unit 10. It is preferable that the image light is corrected to correspond to the curved surface.

By the projection device 101 according to this embodiment, it is possible to provide an image which is in focus to an observer even when an observation position changes.

### Fourth Embodiment

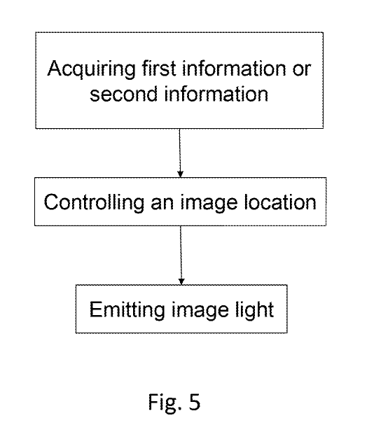

A flow of operation of projection device 100, 101 will be described. FIG. 5 is a flow chart illustrating a performance of a projection device.

The first position acquiring unit 50 acquires first information regarding a position of the table 302. In the case where the second position acquiring unit 51 is used instead of the first position acquiring unit 50, the second acquiring unit 51 acquires second information regarding a position regarding the reflection unit 30 instead of the first information.

And then, the image location of the first emitting unit 10 is controlled. That is, the first emitting unit 10 may move to follow the position of the table 302 or the first optical element 13 may change the focal length by moving an optical component(s), or a part of the optical elements included in the first optical element 13. Therefore an image location is provided on or near the projection unit.

To control an image location, a movement distance of the first emitting unit 10 may be calculated prior to the movement. To control an image location, a difference of focal length may be calculated prior to changing a focal length. To control an image location, appropriate arrangement of an optical component(s) may be found prior to changing the arrangement. And then, the first emitting unit 10 emits image light.

After the first information is acquired and before the image location is controlled, an appropriate focal length may be acquired based on a position of the table 302.

After the first information is acquired and before the image light is emitted, appropriate magnifications of first optical element 13 and second optical element 22, and appropriate brightness of an image light may be found based on a position of the table 302. The first emitting unit 10 may emit an image light based on the appropriate magnifications and brightness.

There is a case where people have a sense called vection when they observe images. People may have this sense even if they are not moving. Picture sickness may be caused because of this sense. It is said that picture sickness is caused because of a gap between motion sense by a physical movement and visual sense by an image. To prevent picture sickness, it is desirable to suppress the gap between the motion sense and visual sense.

In this embodiment, the projection device 100, 101 is configured to project an image of an object which moves to follow the table 302.

By the projection device 100, 101 according to this embodiment, it is able to provide an image which is in focus to an observer even while an observation position changes.

Each of the embodiments was described with specific examples. However, this disclosure is not limited to these specific examples. For example, one of ordinary skill in the art will understand that this disclosure may be implemented using available variations in the specific composition of each element.

One of ordinary skill in the art will also understand that this disclosure may be implemented using combinations of two or more elements from the specific examples.

One of ordinary skill in the art will also understand that this disclosure may be implemented using other optical devices and image display apparatuses.

While certain embodiments have been described, these embodiments have been presented by way of example only, and are not intended to limit the scope of the disclosure. Indeed, the embodiments described herein may be embodied in a variety of other forms; furthermore, various omissions, substitutions and changes in the form of the embodiments described herein may be made without departing from the spirit of the disclosure. The accompanying claims and their equivalents are intended to cover such forms or modifications as would fall within the scope and spirit of the disclosure.

1. A medical imaging apparatus comprising:
a gantry;
a table being movable to go in and out of the gantry;
a first light emitting unit being apart from the gantry and configured to emit image light; and
a reflection unit being configured to reflect the image light toward the table.

2. The apparatus according to claim 1, wherein the
reflection unit has a refractive power.

3. A projection device comprising;
a first light emitting unit being configured to emit image light toward a movable table, the first light emitting unit is configured to change an image location of the image light based on a position of the table.

4. The device according to claim 3, wherein the first light emitting unit is configured to change an image location by moving based on a position of the table.

5. The device according to claim 3, wherein the first light emitting unit includes a lens, and is configured to change an image location by moving a position of the lens.

6. The device according to claim 3, wherein the first light emitting unit includes a plurality of lenses and is configured to change an image location by changing an arrangement of the lenses.

7. The device according to claim 3, further comprising a first position acquiring unit configured to acquire first information regarding a position of the table.

8. The device according to claim 3, further comprising a second position acquiring unit configured to acquire second information regarding a position of a reflection unit arranged between the table and the first light emitting unit on a light path of the image light.

9. The device according to claim 3, further comprising an image signal acquiring unit configured to acquire an image signal, the first light emitting unit light emitting the image light based on the image signal.

10. The device according to claim 3, wherein the first light emitting unit includes a display element and a first optical element.

11. The device according to claim 3, further comprising a second optical element arranged between the first light emitting unit and the table on light path of the image light, the second optical element being movable to follow the table and refract at least a part of the image light.

12. The device according to claim 3, further comprising a reflection unit arranged between the first light emitting unit and the table on light path of the image light, the reflection unit being configured to reflect the image light toward the table.

13. The device according to claim 3, wherein the first light emitting unit is configured to change an image location so that an image height after the change is not less than 90 percent and not more than 110 percent of the image height before the change.

14. The device according to claim 3, wherein the longer a distance between the first light emitting unit and image location is, the higher the luminance of the image light.

15. The device according to claim 12, further comprising a projection unit arranged between the first light emitting unit and the reflection unit on a light path of the image light, the projection unit being smaller than an image to be projected on the projection unit by the first light emitting unit.

16. The device according to claim 12, further comprising a projection unit arranged between the first light emitting unit and the reflection unit on a light path of the image light, the projection unit including a curved surface that is convex with respect to the first emitting unit.

17. A medical imaging apparatus comprising:
a gantry;
a table being configured to be moved into and out of the gantry; and
a first light emitting unit configured to emit image light toward the table, and configured to change an image location of the image light based on a position of the table.

18. An apparatus according to claim 17, wherein the projection unit is smaller than an image formed by the first emitting unit.

19. An apparatus according to claim 18, wherein the projection unit includes a curved surface that is convex with respect to the first emitting unit.
